(19) United States
(12) Patent Application Publication
(10) Pub. No.: US 2018/0310894 A1
(43) Pub. Date: Nov. 1, 2018

(54) Medical Imaging Stabilization and Coregistration

(71) Applicant: The Regents of the University of California, Oakland, CA (US)

(72) Inventors: Jack L. Gallant, Berkeley, CA (US); James S. Gao, Berkeley, CA (US); Alexander Geral Huth, Berkeley, CA (US)

(21) Appl. No.: 15/771,707

(22) PCT Filed: Oct. 31, 2016
(86) PCT No.: PCT/US16/59762

§ 371 Date: Apr. 27, 2018

Related U.S. Application Data
(60) Provisional application No. 62/248,914, filed on Oct. 30, 2015.

(51) Int. Cl. A61B 6/04; A61F 5/37

(52) U.S. Cl. CPC A61B 6/04; A61F 5/3707

(57) ABSTRACT

Methods, systems and devices are disclosed for reducing the motion of a body part during a medical procedure that requires stability of the body part. In one example, the device consists of an object which has been customized so that it conforms to an individual's anatomy. When placed on an individual, the device stabilizes their body during medical imaging or intervention. Virtual targets within the body can be localized as physical locations on the body by adding markers on the device. These markers can be quickly and easily localized across multiple treatment sessions. Because the device is customized for each individual, it is comfortable yet effective.

### FEDERALLY SPONSORED RESEARCH OR DEVELOPMENT

This invention was made with government support under EY022454 (31696-26697-44-EUJLG) awarded by National Eye Institute, and U.S. Pat. No. 1,208,203 (31548-26697-44-EUJLG) awarded by National Science Foundation. The government has certain rights in the invention.

### TECHNICAL FIELD

This instant specification relates to systems and devices for reducing the motion of a body part during a medical procedure that requires stability of the body part.

### BACKGROUND

Keeping patients very still is vitally important during many different medical procedures, including for example medical imaging, radiological intervention, and many types of surgery. As one example, in magnetic resonance imaging (MRI), subject motion causes severe degradation of the image, often forcing scans to be repeated or simply thrown away. Precise and consistent head positioning is also important for longitudinal imaging-based research (e.g. using functional or anatomical MRI) or research programs that require many repeated scans of the same subjects. Similarly, for many radiological and surgical procedures it is important to accurately and repeatably position the patient relative to some known landmarks outside the body. For example, during radiation cancer therapy, a radiation source must be aimed from the outside of the body to precisely strike an internal target. Furthermore, more than 40 sessions may be required to achieve the requisite dosage. Any imprecision in the positioning of the source with respect to the patient across sessions will result in decreased effectiveness (as less total radiation is delivered to the tumor) and increased side-effects (as more radiation is delivered to neighboring healthy tissue).

By way of example to further illustrate the challenges in the art that are addressed by the techniques being disclosed in this document, in functional MRI procedures head motion can cause the deleterious effects of reducing the magnitude of functional signals, creating spurious signals, and biasing estimates of functional connectivity. These problems are compounded by modern fMRI acquisition techniques such as generalized auto-calibrating partially parallel acquisitions (“GRAPPA”) techniques and simultaneous multi-slice (i.e., multiband) techniques, which offer increased scan efficiency at the cost of increased motion sensitivity.

Current methods for reducing head motion include foam padding, inflatable pneumatic pads and bite bars. However, these methods reduce head motion only modestly and they can be very uncomfortable. Furthermore, these methods typically do not improve the consistency of head positioning across imaging sessions. In addition, current methods for minimizing motion and for establishing stereotaxy in radiotherapy and surgery are either only marginally effective (e.g. thermoplastic mesh placed over the face), or they require invasive surgery (e.g. stereotactic frames which screw into the skull).

Another known method of dealing with motion sensitivity is prospective motion correction, where subject motion is tracked in real time using an infrared camera and the scan window is moved to compensate. However, even with prospective motion correction, any motion during a scan would still reduce the efficacy of magnetic field shimming, resulting in decreased image quality. Furthermore, motion that occurs within a slice acquisition causes shearing of that slice along with misalignments in subsequent slices. Thus, prospective motion correction has limitations in being able to fix motion-related artifacts.

Another example of an effort to immobilize subjects during a study was by means of a personalized plaster cast which was attached to a head coil of a MRI machine. While effective in reducing subject motion, the method has drawbacks in that it requires substantial effort, time, and skill to produce the cast. Furthermore, this method requires extensive subject access and participation, elements which may not be available in typical experimental paradigms.

### SUMMARY

In this document, we present methods, systems and devices for reducing the motion of a body part during a medical procedure that requires stability of the body part, as set forth in the claims, description and figures.

For example in one exemplary aspect, this document discloses a new device that stabilizes the body with respect to some known location. This device is custom designed for each individual person and it can be manufactured quickly using rapid prototyping technologies. In the case of medical imaging of the head, this device consists of a custom helmet (e.g., case, device insert) designed to fit precisely into the imaging device. This helmet dramatically reduces head motion during imaging and it improves head placement consistency across imaging sessions. The device is produced by an integrated rapid prototyping system that produces an enclosure that is customized to fit snugly over the head of each individual patient or participant. An anatomical model of the head is used to optimize the helmet for each individual. The anatomical model can be derived from an anatomical MRI, three-dimensional scanning, or CAD modeling. The device can be made out of a variety of materials such as polyurethane foam or polyactic (PLA) plastic. Finally, the digital model can be modified so that the device can carry imaging components such as gradient coils, surface electrodes, optrodes, or other sensors. It can be designed to integrate with any peripheral that requires consistent or optimal placement with respect to the body. This device dramatically reduces subject motion, improves signal quality, and improves consistency of patient placement and co-registration across sessions and between imaging modalities.

More generally in one aspect, this document discloses a body positioning device, which may be referred to as a “case,” (e.g., device insert) for use in a medical procedure. The case device includes a body-worn structure having an inner surface with a shape configured to complement a shape of a body part to be subjected to a medical procedure. The case device may also include a connection structure adapted to mate with structure of a machine for performing the medical procedure so as to register the position of the body part wearing the body-worn structure with the machine for performing the medical procedure. In some embodiments, the method, systems and devices do not include such a connecting structure, as the device itself may be adapted to be self-stabilizing with respect to the machine. It is contemplated that the case may be made out of a variety of known materials such as polyurethane foam, polystyrene foam, and hard plastic as just three examples, as discussed below, and it is further contemplated that various new materials may be introduced in the future that may achieve objectives in accordance with the teachings in this document, and therefore are also contemplated as being within the scope of subject matter being described herein.

In another general aspect of the teachings provided in this document, a method is disclosed utilizing an additive or subtractive manufacturing procedure for forming a body positioning device for use in a medical procedure. The method includes capturing data for a shape of a body part to be subjected to a medical procedure involving a machine that performs the medical procedure, and using the captured data in the performance of a milling procedure upon a source material (or alternatively in the performance of an additive manufacturing procedure) to form a body positioning device having an inner surface with a shape configured to complement a shape of a body part to be subjected to a medical procedure. The body positioning device may additionally be manufactured to include connection structure adapted to mate with structure of a machine for performing the medical procedure so as to register the position of the body part within the body worn structure with the machine for performing the medical procedure. The method may also include a process in which measurement data for the machine for performing the medical procedure is used to create a fixturing device for subtractive manufacture of a case. The inner surface can be 3D scanned or otherwise measured to capture measurement data, and the captured data can be used in the performance of a milling procedure upon the source material to form the body positioning device having an outer surface with a shape configured to complement the shape of the machine used to perform the medical procedure.

In yet another general aspect of this disclosure, a system is disclosed for forming a body positioning device for use in a medical procedure. The system comprises a receiver adapted to receive image data taken for a patient that is descriptive of a shape of a body part for the patient, and processing circuitry adapted to transform the stored body part shape image data into computer readable instructions to be provided to an automated manufacturing machine, such as a milling machine or an additive manufacturing machine such as a 3D printer, to produce a body positioning device having an inner surface with a shape configured to complement the shape of the body part for the patient. The processing circuitry may be further adapted to produce computer readable instructions to be provided to the automated manufacturing machine to manufacture the device to also include a connection structure adapted to mate with structure of a machine for performing the medical procedure so as to register the body part within the body-worn structure with the machine for performing the medical procedure. The system may also include a 3D scanner configured to collect measurement data for the machine for performing the medical procedure, and a fixture adapted to receive the machine measurement data, wherein the computer readable instructions for manufacturing the connection structure are based on the collected machine measurement data.

In another exemplary aspect specific to the field of fMRI procedures, this document discloses a case device, made for example of polyurethane foam, polystyrene foam, or a rigid plastic material that fills the space between the MRI head coil and an individual subject's head. The case is designed so that it conforms to the inside surface of the head coil and the outside surface of the subject's head. A fused-deposition three-dimensional (3D) printer may be used to manufacture the case out of polylactic acid (PLA). The resulting case constrains head motion more effectively than padding, and it substantially reduces the effort required from the subject to keep still. Furthermore, the rigid plastic case does not compress over the duration of an experiment, reducing subject drift. In this method, a separate case is manufactured for each individual subject. The cost of this is likely worthwhile when subjects will be used in multiple experiments, when each subject will participate in many scanning sessions or when subject motion is a particular problem or concern. Further, recent advances in the field of 3D printing has made individual manufacture of these cases a relatively economical option given the cost of subject disqualification due to motion.

As discussed in the background discussion above, two factors that impact signal quality in fMRI procedures are subject motion, and placement of the subject within the magnetic field. The devices, methods and systems that are disclosed in this document address both of these issues. They allow consistent placement of the subjects head within the MRI coil and within the magnetic field across scan sessions, and minimize motion of the head during each scan session. They also produce a case device that is also quite comfortable to wear. For these reasons for example, it is expected that this case device will find wide use in MRI studies, especially those involving long or multiple scan sessions.

In addition, other imaging devices such as MRI receive coils can be integrated with the helmet. This reduces the distance between the receive coils and the brain, and so increase signal quality and sensitivity.

Also, the device can also be used to improve the stability of other methods for measuring physiology or anatomy, such as electroencephalography (EEG), magnetoencephalography (MEG), functional near-infrared spectroscopy (fNIRS), positron emission tomography (PET), nuclear imaging, ultrasound sonography, or X-ray computed axial tomography (CAT). Signals measured using these methods are also limited by the motion of the head relative to the sensors, and by the difficulty of obtaining consistent sensor placement across sessions. By integrating EEG sensors, optrodes or other sensors directly into the device, relative motion can be minimized and consistency can be maintained. This can improve signal quality significantly.

Also, the teachings disclosed in this document enable specialized devices to be produced for radiation therapy. In such an application, patients can be placed reliably in the same location across multiple sessions throughout an entire treatment regimen. The therapy target can be located virtually in MRI images, and a slot can be built into the device to hold the radiation source. This will allow smaller and more consistent placement of radiation sources to reduce damage to neighboring tissue.

In another application of the teachings of this document, specialized devices can be produced for frameless stereotactic surgery. A surgery (e.g., brain biopsy) can be planned using radiological images of the patient. Then a helmet can be manufactured with precisely placed markings or holes showing or limiting where the surgical instruments will be inserted into the skull. In a further application of the teachings in this document, custom orthopedic casts can be manufactured to stabilize fractures. These casts can easily be placed and removed without extensive training. In another application, diabetic foot ulcers and other pressure-related ulcers can be treated with custom devices which are form fit for the body. Pressure ulcers have been shown to heal much faster when weight is distributed and direct pressure on the ulcers are alleviated.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an example of a stabilization and coregistration system.

FIG. 2 illustrates an example of a process for generating a case device for a stabilization and coregistration system.

FIG. 3 is conceptual illustration of an example process for reconstruction of a scalp surface of the subject's head.

FIG. 4 shows another view of the example of the case device.

FIG. 5 shows the example case device as it is being shaped by an example manufacturing apparatus.

FIG. 6 shows an example of a completed case device.

FIGS. 7A and 7B show another example of a completed case device.

FIG. 8 shows the results of several studies of patient movement with and without a case device.

FIG. 9 shows the results of several studies of patient movement with and without a case device.

FIG. 10 is a block diagram of an example system for manufacturing a case device.

FIG. 11 is a flow diagram of an example process for manufacturing a case device.

FIGS. 12A-12D show an example case device in use.

### DETAILED DESCRIPTION

By way of overview, in one embodiment the device consists of an object (e.g., case device, device insert) which has been customized so that it conforms to an individual's anatomy. When placed on an individual, the case device stabilizes their body during medical imaging or intervention. In addition, virtual targets within the body can be localized as physical locations on the body by adding markers on the case device. These markers can be quickly and easily localized across multiple treatment sessions. Because the device is customized for each individual, it is comfortable yet effective. For example, a customized helmet can be designed to hold a subject head still for magnetic resonance imaging (MRI). Such a device would keep the subject still, thereby improving signal quality and reducing total scan time. The subject can be reliably placed in the same location, facilitating tracking across multiple scanning sessions.

In terms of overview and general description of the method of manufacturing the case device, the method may include the following general steps: (1) a 3D model of the subject's body is acquired to construct a CAD model of the body; (2) a 3D model of the medical device in which the case device will fit is built using traditional CAD procedures or 3D scanning; (3) constructive solid geometry algorithms or other 3D model manipulation algorithms are used to subtract the body model from the medical device model, which produces a solid structure that fills the space between the body and the medical device; (4) Additional areas may be cutout from the solid structure for eyes, ears, neck, mouth, or additional medical devices; and (5) a rapid prototyping method is used to produce the device either using fused-deposition 3D printing or rapid CNC milling of a variety of materials.

In one implementation, the device consists of a foam or plastic structure that is customized for each individual person. The exterior surface is designed to rigidly lock to a reference point, while the interior comfortably cradles the person's individual anatomy. The exterior surface can be seamlessly integrated with medical imaging or surgical systems to provide fast and easy stabilization and coregistration to the reference frame. In one example where the case device is produced using a milling or other subtractive manufacturing process, a particularly advantageous starting material to use to form the case device is polyurethane foam or other similar rigid foams. Such a material is advantageous because it has relatively little give in it, and thus minimizes movement of the subject just as a hard plastic material would, but a polyurethane foam or similar material is very inexpensive and easy to use in a milling process. Importantly in this regard, polyurethane foam by its nature is easy to cut, making the milling process of subtracting material to produce an end product quick and easy to perform, unlike many other hard materials where the milling process may take hours to perform. Having a milling process that can be done very quickly as is the case with polyurethane and other similar rigid foam materials is particularly important for many medical applications where a patient may be sized for the device, and then shortly thereafter subjected to a medical procedure in which the device is used.

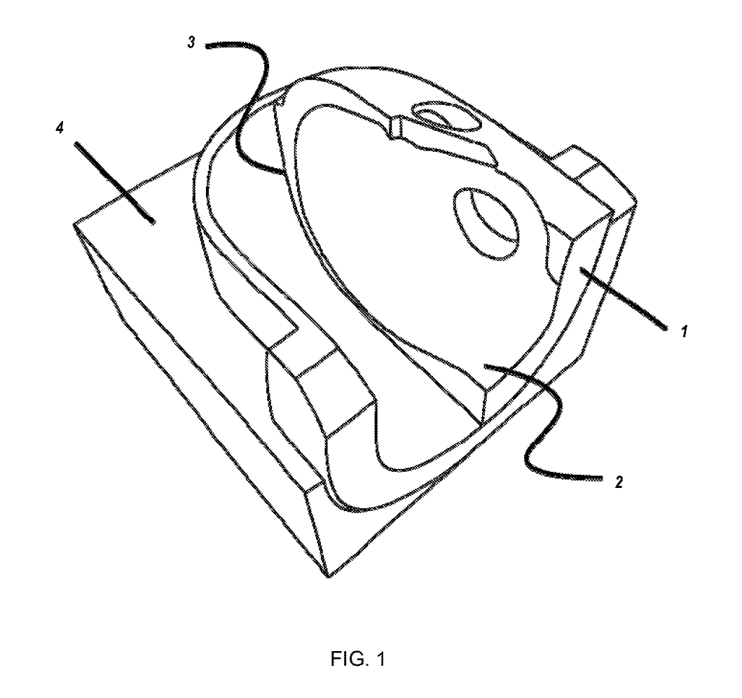

To understand an example of how the device may be constructed, consider a case device designed to optimize data acquisition in fMRI brain research, as shown FIG. 1. In this instantiation, the device consists of a case device 1 (e.g., a helmet, device insert) (one half shown) having an inner surface 2 that is adapted to an individual head, and having an exterior surface 3 that is adapted to an interior surface of an MRI head coil 4.

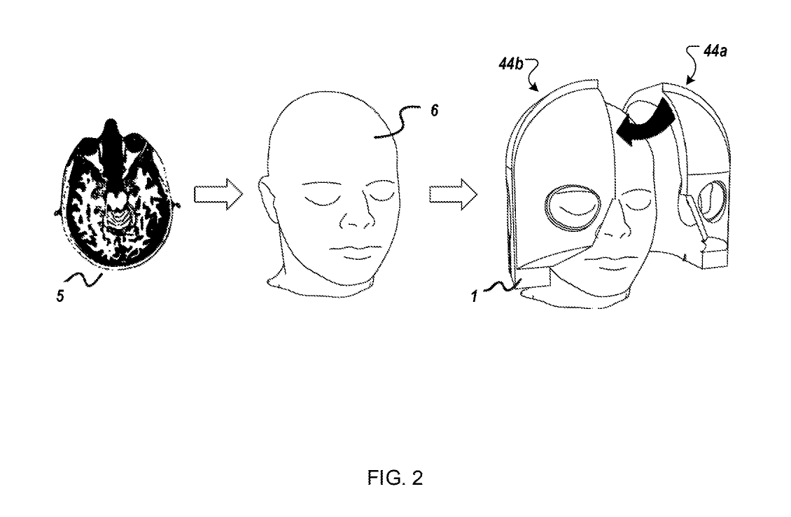

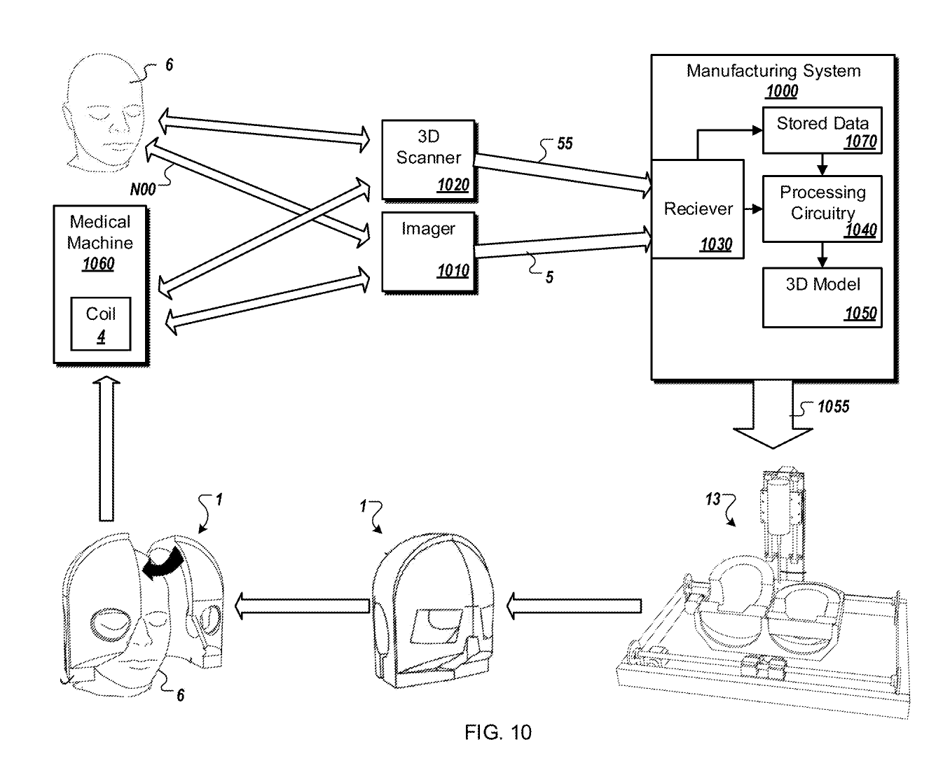

FIG. 2 illustrates an example of a process used to generate the example case device 1 of FIG. 1. FIG. 10 is a block diagram of an example system 1000 for manufacturing the example case device 1. First, imaging scans 5 from a medical imager 1010 (e.g., a CT or other tomographic imager) and/or topographic scans 55 from a 3D scanner 1020 descriptive of a shape of a body part for the patient, such as a subject's head 6, are received at a receiver 1030 adapted to receive the imaging scans 5 and/or the topographic scans 55. The received data is processed by a processing circuit 1040 to produce a 3D model 1050 of the subject's head 6, including the detailed surface and shape of the subject's head 6. The processing circuit 1040 is adapted to transform the image data into a collection of computer readable instructions 1055 (e.g., CNC instructions) to be provided to an automated milling machine 13 to mill a source material into a body positioning device having a milled inner surface with a shape configured to complement the shape of the body part for the patient.

The instructions 1055, based on the 3D model 1050 of the subject's head 6, are utilized to produce the case device 1 such that the case device 1 has an inner surface (e.g., the inner surface 2 shown in FIG. 1) that complements the shape and contour of the subject's head 6. In the example shown in FIG. 2, the case device 1 is a two-part design, having a left half portion 44a and a right half portion 44b, enabling the user to put the case device 1 on so that it surrounds the subject's head 6. In addition, the case device 1 in this example also includes an outer surface 3 that has a shape so that it rigidly and fixedly mates with a structure of the MRI head coils 4 although there are various different configurations of head coils and medical machines to which the techniques described in this document would apply.

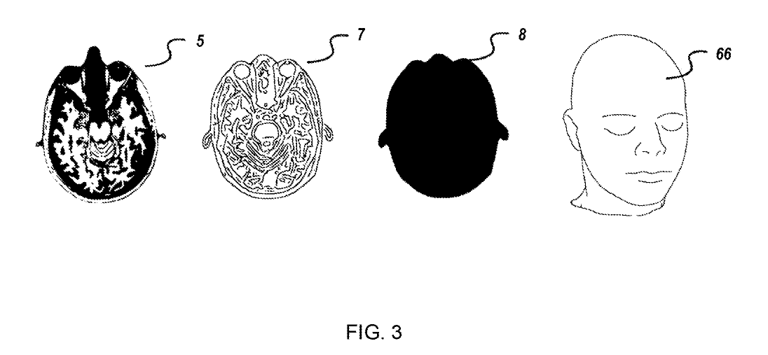

FIG. 3 is conceptual illustration of an example process for reconstruction of a scalp surface of the subject's head 6. In some embodiments, the case device 1 may be constructed by means of a subtractive manufacturing process that can include the following steps. First, an MRI or 3D scanner (e.g., the imager 1010 and/or the 3D scanner 1020 of FIG. 10) can be used to capture imaging scans 5 and/or topographic scans 55 of the subject's head 6. These imaging scans 5 and/or topographic scans 55 can be used to reconstruct the geometry (e.g., model) of the subject's head 6 such as the 3D model 1050. A case, such as the case device 1 that matches precisely the shapes of both the subject's head (as well as the head coil as described below) may use a high quality 3D model of both the subject's head 6 and the coil. In one example, the NextEngine 3D scanner HD (NextEngine, Santa Monica, Calif., USA) may be used to obtain a 3D model of the subject's head 6 and the MRI head coils. For example, to obtain a 3D model of the subject's head 6, a T1-weighted anatomical MRI can be acquired on a Siemens TIM Trio scanner with a 12 channel coil. The reconstruction of a scalp surface is illustrated in FIG. 3.

An imaging scan 5 may be smoothed and median filtered to reduce noise. A canny edge filter can be performed on the smoothed imaging scan 5 to produce a filtered image 7. In some implementations, holes in the edges of the filtered image 7 can be closed with binary dilation followed by binary erosion. A flood fill algorithm is performed on the filtered image 7 to generate a scalp volume 8. A marching cubes algorithm is used to generate a surface reconstruction model of the subject's head 66. In some implementations, Laplacian smoothing and quadric decimation can be applied to smooth and simplify the surface reconstruction model 66. In some implementations, the surface reconstruction model 66 can be expanded (e.g., by about 2 mm) along each vertex normal, for example, to account for hair and pulsatile expansion of the subject's head 6.

Alternatively, in order to generate the surface reconstruction model 66 without the use of an MRI machine, the subject's head 6 can be scanned with the 3D scanner 1020. In some implementations, subject may wear a tight-fitting cap in order to flatten hair. The 3D scanner is used to generate a millimeter-accurate model of the subject's head 6 for use in subsequent steps. Additional processing steps including poisson surface reconstruction and smoothing are used to eliminate self-intersections and topological errors in the surface, which improves the function of solid geometry subtraction.

The second step of the subtractive manufacturing method is that the MRI head coil 1 is 3D scanned and recreated in CAD software. Referring to FIG. 10, a portion of a medical machine 1060 (e.g., an MRI having the MRI head coil 1) can be imaged with the medical imager 1010 and/or the 3D scanner 1020 to collect imaging data 5 and/or topographic data 55 that can be provided to the receiver 1030. In yet another implementation, CAD software may be used to model the inside surface of the MRI head coil 4. In some implementations, the process of scanning the subject's head and/or the scanning of the machine may be performed with a portable (e.g., handheld) scanning tool, such as a purpose-built 3D scanner or an imaging application executed on a handheld computer or smart phone. In yet another implementation, CAD software may be used to model the inside surface of the MRI head coil 4, and such a model may be stored as a collection of stored data 1070. The imaging data 5, the topographic data 55, or the stored data 1070 can be used to produce the 3D model 1050 such that the 3D model 1050 includes a shape of a connecting structure that mates with the MRI coil 4. The computer readable instructions 1055 can determine instructions that, when performed by the milling machine 13, cause the case device 1 to have the shape of the connecting structure.

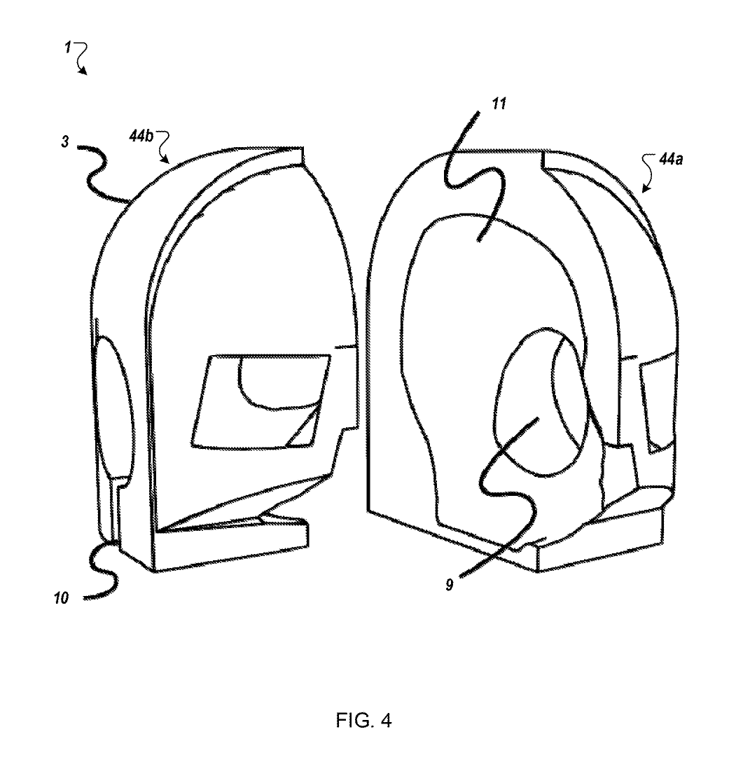

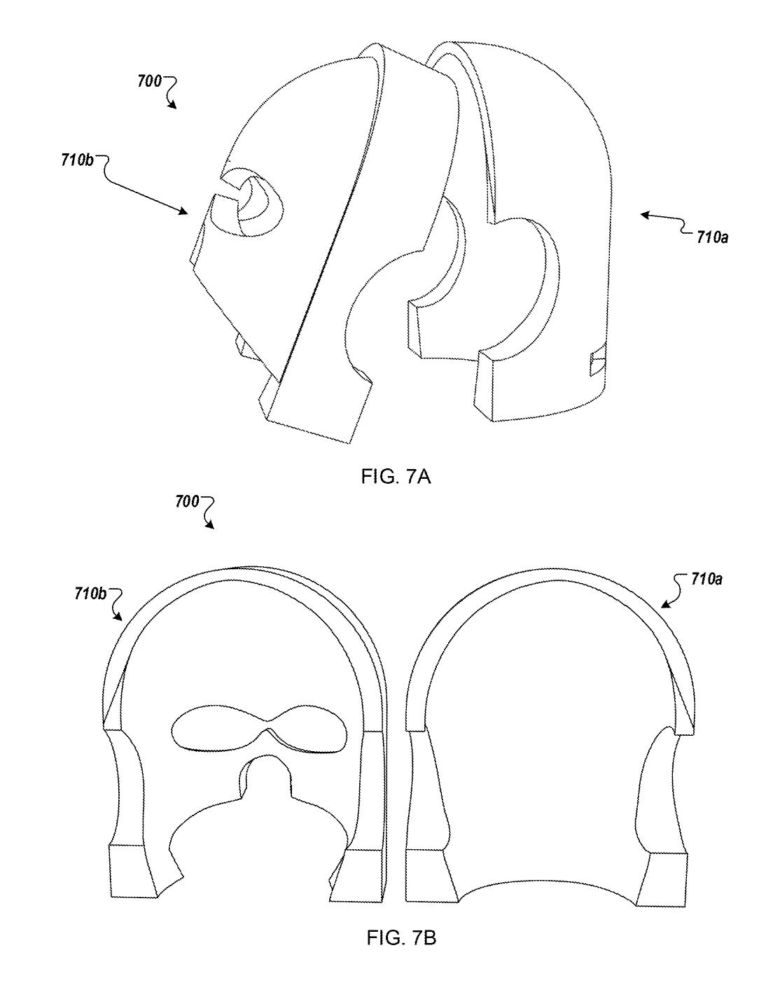

FIG. 4 shows another view of the example of the case device 1 of FIGS. 1 and 2. The third step of the manufacturing method is to produce a master mold of the head coil with cutouts 9 for bodily protrusions, such as the subject's eyes, ears, and neck. This master mold is formed such that casts made from it have outer surfaces 3 that take on the shape of the medical device to be interfaced with, such as the MRI head coils 4. In some embodiments, the master mold can be modified to add indents, receptacles, conduits, and cutouts 10 for insertion of ancillary hardware. In some implementations, a large cavity 11 can be included, for example, to reduce processing time during milling. In some embodiments, the master mold can be configured such that the case device 1 includes a split, for example, to allow entry of the subject's head 6. As described previously, the case device 1 is formed in two parts, having the left half portion 44a and the right half portion 44b. Referring back to FIG. 1, in use, the left half portion 44a can be placed over the left side of the subject's head 6 and the right half portion 44b can be placed over the right side of the subject's head 6. In some embodiments, the case device 1 can be formed in two or more portions in other arrangements, an example of which will be discussed later in the descriptions of FIGS. 7A and 7B.

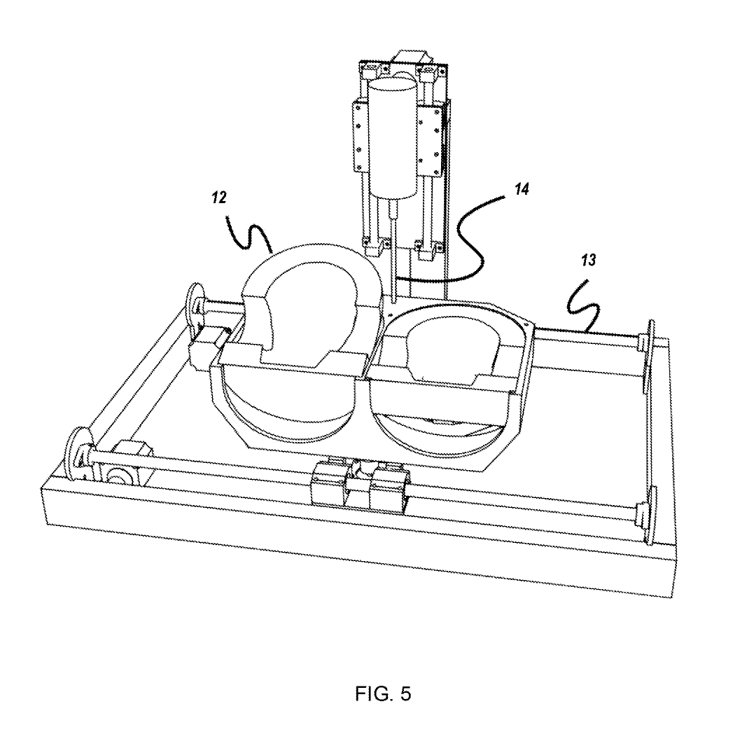

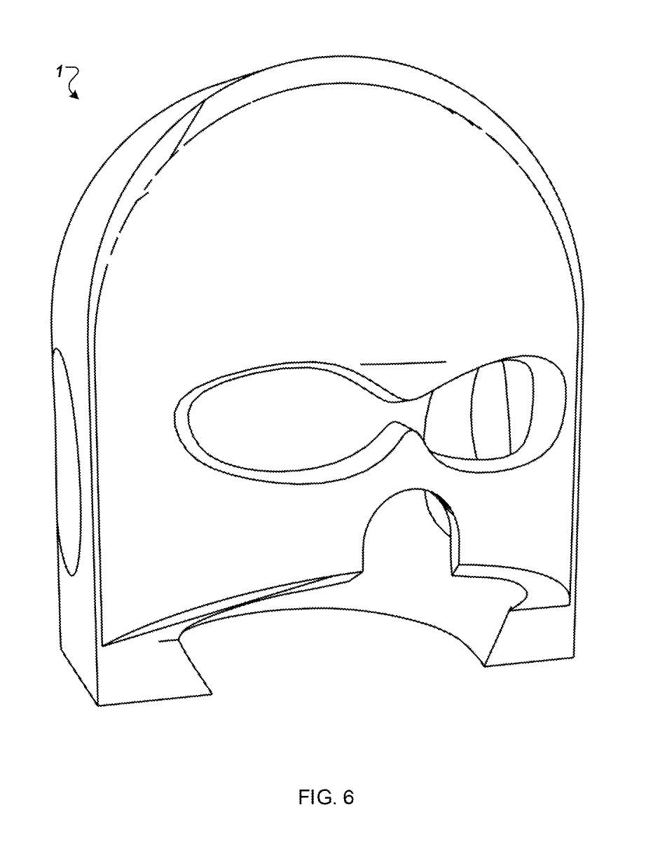

FIG. 5 shows the example case device 1 as it is being shaped by an example manufacturing apparatus. In the fourth step in the manufacturing process, helmet blanks 12 are cast from the master mold using polyurethane or polystyrene foam. In a fifth step, an integrated software suite (e.g., executing on the processing circuitry 1040 of FIG. 10) intakes the surface reconstruction model 66 of the subject's head 6, automatically aligns the surface reconstruction model 66 with the cutouts 10 for the eyes and ears, and generates a highly optimized toolpath which closely follows the surface of the subject's head 6 based on the surface reconstruction model 66. Sixth, the foam blank 12 is inserted into a custom computer numerical control (CNC) mill 13 equipped with a ball nose end mill 14 which rapidly carves out the surface of the surface reconstruction model 66 from the blank 12. In some implementations, CNC technology such as the CNC mill 13 can generate the case device 1 in just a few minutes, allowing production during a single imaging session. An example of a completed case device 1 is shown in FIG. 6.

Another example of a completed case device is shown in FIGS. 7A and 7B. In some implementations, the milling process performed by the CNC mill 13 can be done in two general stages. In a first stage, the outer surface 3 of the case device 1, which mates with the MRI head coil 4, can be milled from two blocks of material (e.g., extruded polystyrene foam) for front and back (or left and right) halves. In a second stage, the material can be flipped and placed in a milling fixture, and the inner surface 2 (e.g., which mates with the subject) is hollowed out. In some implementations, a proprietary processing pipeline can be used to generate a substantially constant-scallop tool path which optimizes for production speed and minimizes milling artifacts. In some examples, since the exterior of the case device 1 is constant across subjects for a particular medical machine, “blanks” made from the selected material can be manufactured with specific outer surfaces 3 and generalized inner surfaces 2 in advance and stockpiled to allow rapid production of subject-specific cases (e.g., by performing the second phase on a blank previously made in the first phase). In some examples, a full milling cycle for both blank halves can be accomplished in about 40 minutes, and a new subject-specific case device 1 can be produced from scan to fitted device in under about 60 minutes.

In some implementations, other materials and manufacturing techniques can be used. For example, the case device 1 may be printed out of polylactic acid (PLA) using a fused deposition 3D printer. Alternatively, a scanning laser sintering device can generate the case device 1 from nylon powder, or other suitable material. In another example, a stereolithograph, or any other appropriate manufacturing device can be used to form the case device 1 from resin or any other appropriate material.

PLA may be used as the material because it has little to no MRI signal, and its low shrinkage results in high fidelity printed models. For this additive production method, additional steps may be taken during modeling to produce the case device 1. For example, in order to generate the full case device 1, a model of the interstitial space between the subject's head 6 and the MRI head coil 4 can be produced. This model may be obtained by boolean constructive solid geometry operations between the head model and the coil model. This may involve taking the difference of the inside surface of the MRI head coil 4 and the subject's head 6. Again as mentioned, extra holes 10 may be created to make room for the ears, nose, eyes, and mouth, and for additional hardware such headphone wires and lenses for refractory correction. The case device 1 can be split into multiple pieces, for example, to reduce overhang and facilitate printing, and the model is then printed. The pieces may then be fastened together with nylon bolts or other appropriate fasteners, or may be glued together (e.g., with superglue or other appropriate adhesive).

FIGS. 7A and 7B show another example case device 700. The case device 700 is substantially similar to the case device 1, except the case device 700 is split into a front half portion 710a and a back half portion 710b (e.g., as opposed to being split into left and right half portions 44a and 44b as in the example of the case device 1). The case device 700 can be split such that the front (e.g., face) portion 710a and the back (e.g., back of the head) portion 710b are separate pieces. For example, in some applications only the back portion 710b of the case device may be manufactured and/or used for a particular procedure, e.g., a process in which the patient would lay down on his or her back and rest his or her head in a case device or portion that positions the patent's head. In another example, the patent may also undergo a different procedure in which the front portion 710a of the case device 700 is assembled to the back portion 710a to position the subject's head 6 during a different process.

In some embodiments, the case devices 1 may be formed with two or more layers. An inner layer may include the inner surface 2, and an outer layer may include the outer surface 3. The outer layer may include an inner surface that is sized and includes registration features that are common to all such outer layers, and the inner layer may include an outer surface that complements common size and registration features. As such, the inner layer registers to the subject's head 6 or other body part, the outer layer registers to the MRI head coil 4 or other device, and the inner layer fits within the outer layer such that the inner and outer layers register to each other. As such, a single outer layer may be constructed for each MRI head coil 4 or other device, and a single inner layer may be constructed for each subject (e.g., instead of constructing a separate complete case device 1 for each imaging device that each subject may use). For example, for five patients and five machines, 25 single layer case devices may be constructed (e.g., 5 heads×5 machines=25 case devices), or ten multi-layer case devices may be constructed (e.g., 5 inner layers+5 outer layers=10 case device combinations). In the subsequent disclosure below, any discussion with regard to the case device 1 can also be applied to the case device 700 as well.

In some embodiments, such as when the case device 1 is used for neuroimaging applications, additional neuroimaging hardware (e.g., embedded MRI coils, optrodes for fNIRS) can be inserted into appropriate receptacles in the case device 1. In some examples, such fMRI head stabilization devices can produce a significant reduction in subject motion, and a substantial improvement in imaging signal quality.

Advantages

The devices, methods, and systems described herein may have many advantages, including the following. First, the case device 1 can be built out of economical materials such as PU foam, other expanded polymer materials, or standard polymer resins such as PLA plastic, using a rapid prototyping method such as additive 3D printing or subtractive CNC milling. Second, the case device 1 can reduce subject motion within imaging sessions. This can improve signal quality within sessions and can improve alignment of the subject's head 6 across sessions.

Third, the case device 1 can improve the consistency of subject placement across sessions. Existing systems allow positioning error up to 4 mm, whereas the case device 1 can reduce this error to 2 mm or less. Fourth, the case device 1 can reduce the need for post-processing to achieve co-registration across sessions. Many existing systems require a complicated and error prone setup and co-registration process (e.g., optical stereotactic systems). Fifth, the case device 1 can provide a simple and accurate means of aligning data generated by different imaging modalities in the same subject.

Variations

Several extensions and variations are possible. For example, the case device 1 can be modified to include channels for fitting the MRI head coils 4 directly into the case device 1 itself. These coils can be manufactured separately and relatively economically, for example by using screen printing technology, and then inserted directly into the case device 1. In such examples, the coils can be positioned much closer to the brain, and by doing so significantly increase signal quality. Furthermore, such embodiments can reduce or eliminate the need for the separate MRI head coil 4 entirely.

In some embodiments, the case device 1 may be modified to include other devices for measuring brain activity, such as electrodes for electroencephalography (EEG), or optical sources and detectors for functional near infrared spectroscopy (fNIRS).

In some embodiments, the case device 1 can be manufactured to consistently and accurately place a patient with respect to a radiation source during radiation therapy. For example, imaging data can be utilized to target the radiation; virtual targets can be translated to physical locations quickly and easily in order to minimize damage to surrounding tissue due to inaccurate placement or patient motion.

Also, the case device 1 could be used to register fiducial markers during surgical planning. For example, surgical targets can be coregistered with physical locations using disposable case devices 1.

In some implementations, multiple case devices may be produced to have substantially the same interior shape (e.g., for a particular patient), but with differing exterior shapes (e.g., to register the patient with multiple different machines). For example, a patient can be to be transferred from MRI, to CT, to radiation therapy machines all within the same positional reference frame (e.g., position transfer).

In some embodiments, the case device 1 may also be customized for other body parts such as knees or feet. For example, diabetic ulcers may be reduced with custom made boots. In other examples, customized orthopedic casts may be fabricated to fit and stabilize any part of the body. In some embodiments, the case device 1 may also be customized for non-human body parts (e.g., veterinary medical procedures) and other object (e.g., forensics of inanimate objects). Other examples may be contemplated as well.

Example and Test Results

MRI Methods

Two subjects were scanned to test the effectiveness of a case device as described herein using an experimental paradigm designed to measure effects on functional signal-to-noise ratio (fSNR). It was found that the case device substantially reduced motion parameters estimated by retrospective motion correction and it increased fSNR in much of the brain. These results suggest that the 3D-printed case device could drastically reduce the problems of head motion in fMRI studies.

As further detail of the experimental results, fMRI data were collected on the same scanner used in the manufacturing process, specifically using a 32-channel Siemens volume coil. This coil was used for functional imaging because it provides much higher functional SNR (fSNR) than the 12-channel coil. Functional scans were collected using gradient echo EPI with TR=2.0045s, TE=34.5 ms, flip angle=74 degrees, voxel size=2.24×2.24×4.1 mm (slice thickness=3.5 mm plus 18% slice gap), matrix size=100×100 and field of view=224×224 mm. Twenty-four axial interleaved slices were prescribed to cover the majority cortex for the subjects. A standard fat-saturation sequence was used to suppress signal from scalp fat.

In terms of the fMRI experimental methods, to test the effect of the case device on head motion and fSNR, subjects were shown a 2-minute movie sequence (with sound). This movie consisted of 10-15 second clips assembled from various creative commons licensed videos downloaded from Vimeo.com. Throughout the scan the subject maintained fixation on a small point superimposed at the center of the screen. Scanning took place in two separate sessions separated by two days. In the first session, subjects was first scanned five times while watching the movie without the case. The subjects then came out of the magnet in order to put the case in place and five more movie scans were then acquired with the case. In the second session this procedure was reversed so that the first five scans were with the case and the last five without.

Visual stimuli were presented using an MR-compatible Avotec projector (Avotec, Stuart, Fla., USA) mounted behind the scanner and projecting onto a screen mounted inside the bore of the magnet. The stimuli subtended approximately 24×24 degrees of visual angle. Auditory stimuli were presented using Sensimetrics S14 in-ear piezoelectric headphones (Sensimetrics, Malden, Mass., USA). A Behringer Ultra-Curve Pro hardware parametric equalizer was used to flatten the frequency response of the headphones based on calibration data provided by Sensimetrics.

Subjects

Functional data were collected from two male subjects (both age 28). Subjects were healthy and had normal hearing and vision. Both subjects are experimenters with extensive experience in the scanner and both remain quite still even without the case.

fMRI Data Analysis

Rigid-body motion parameters (translation and rotation) were estimated for each volume in each functional run using the FMRIB Linear Image Registration Tool (FLIRT) from FSL 5.0. All volumes in each run were then averaged to obtain a high quality image. FLIRT was also used to automatically align this average image for each run to a template image collected in a separate scanning session. The cross-run transformation matrix was then concatenated to the motion-correction transformation matrices obtained using MCFLIRT, and the concatenated transformation was used to resample the original data directly into the overall template space. Low-frequency voxel response drift was modeled as a quadratic polynomial and then subtracted from the signal. The remaining signal for each voxel was then z-scored across time within each scan.

To measure fSNR, the mean explainable variance was computed separately for each voxel in each of the two conditions (i.e. with and without the case device). This was done by computing the fraction of variance in each voxel timecourse for each individual scan that could be explained by the mean timecourse of that same voxel across all ten scans. This metric ranges between 0 when the voxel timecourses for each scan are independent, and 1.0 when a voxel has exactly the same timecourse on every scan. Statistical significance of fSNR was estimated by randomly selecting repeats from the two conditions, computing their explainable variance and taking their difference. This procedure was repeated for 100 bootstrap samples, and the p value for the two actual difference was computed on a per voxel basis.

Results

To determine whether the case device reduced head motion, we compared estimated motion parameters with and without the case present. Both subjects tested here are highly experienced experimenters who remain quite still in the scanner even without the case. However, examination of the estimated translation and rotation parameters shows that the case significantly reduced the amount of steady motion for one subject. The second subject had exceptionally good motion parameters, but showed a large non-intentional muscle twitch in one trial. Total root-mean-squared (RMS) motion was significantly reduced for all motion parameters except for yaw-rotation in subject 1. RMS motion was significantly reduced in roll rotation and x-axis translation in subject 2. In conclusion, the case dramatically reduces motion to less than 0.25 mm translation in any direction and less than 0.15 degrees rotation for any subject whose motion exceeds these parameters.

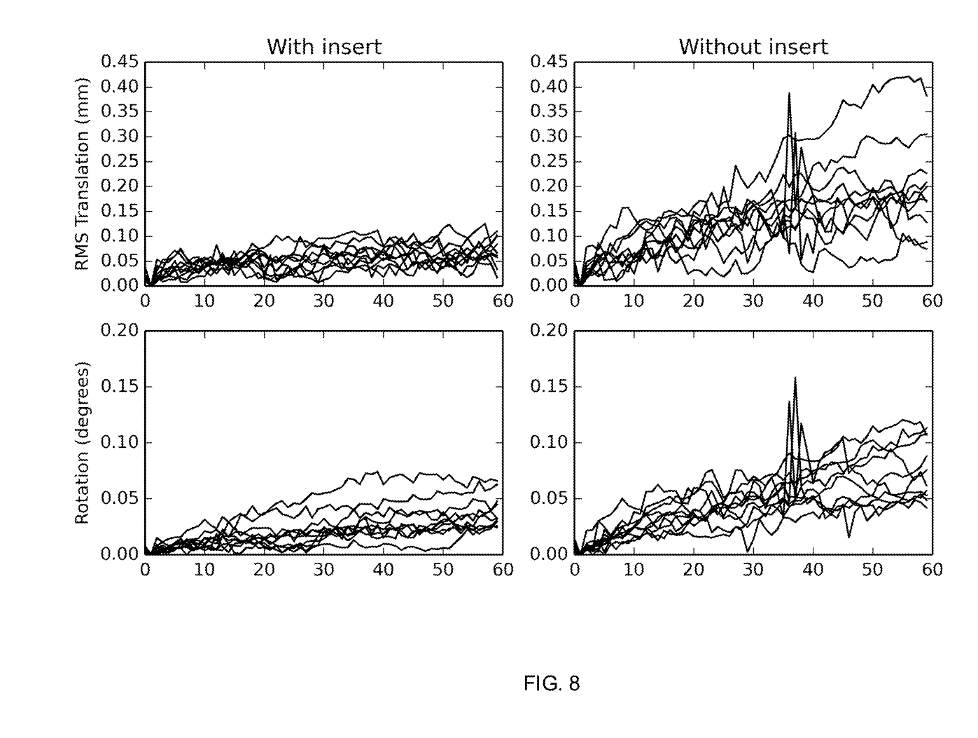

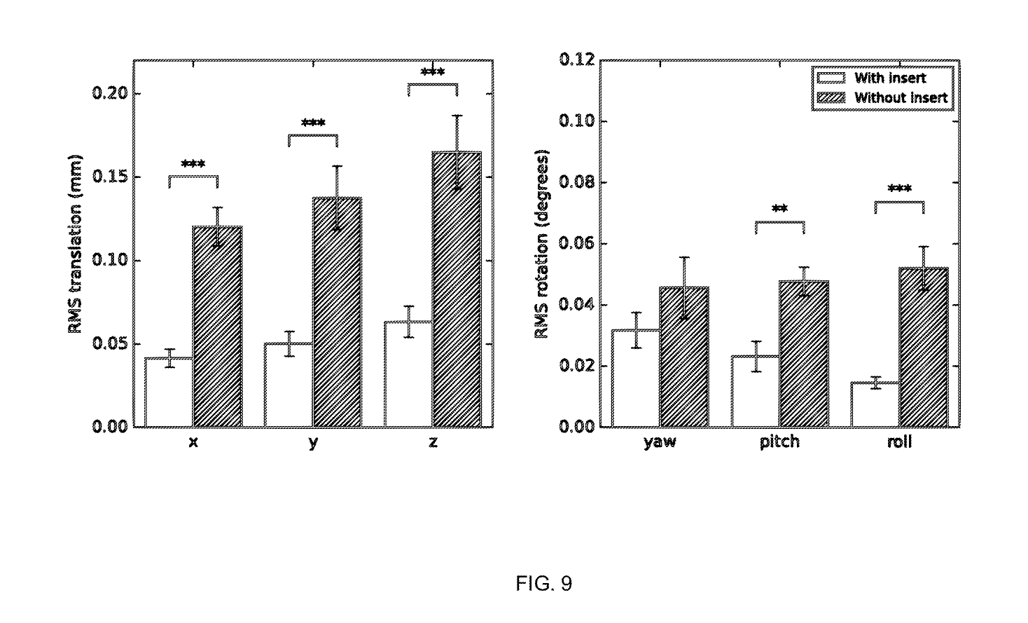

Next there was compared estimated voxel-wise functional SNR (fSNR) values between the two conditions. For the subject who moved relatively more during the scan, the case significantly improved fSNR in approximately 15% of the voxels across the cortex (p<0.05). Even for the subject who moved relatively little in total, fSNR was significantly improved in 10% of voxels across the cortex. Thus, the case device improves fSNR even in compliant subjects who move very little. Results are shown in FIGS. 8 and 9.

Discussion

Thus it is shown that subject head motion during fMRI scanning can be reduced substantially using the techniques described in this document including the use of a manufactured personalized case device to stabilize the head. This case device, which is custom designed and manufactured for each subject, completely fills the space surrounding the subject's head and so makes head motion nearly impossible. Preliminary tests showed that the case significantly reduces motion and increases functional SNR.

The use of a case device in accordance with the techniques described herein substantially improves head stability within a session, as shown in FIGS. 7 and 8.

In addition, the design and manufacturing process may be largely automated and require little human intervention.

The ability to rapidly design and manufacture personalized case devices also opens up many other possibilities for future improvement of MRI data acquisition. For example, it is currently difficult to perform concurrent fMRI and transcranial magnetic stimulation (TMS) because the TMS coils are positioned precisely over the subject's scalp and then locked firmly into place. This problem might be solved by designing case devices with mechanisms with slots to hold the TMS coils in particular positions.

In addition, radiofrequency (RF) transmit inhomogeneity is a severe problem in ultra-high field fMRI (7+ Tesla). This problem can be reduced by surrounding the subject with dielectric pads made of materials such as calcium titanate, but these pads are difficult to position accurately and consistently. In some embodiments, this might be at least partly solved by designing voids in the case device that can be filled with dielectric material, giving improved consistency and a control over the placement of the dielectrics. Since these cases are generated using a high resolution anatomical scan, finite element modeling could also be used to precisely align additional hardware and model how they interact with the tissues in the subject's head.

Another potential application of the approach proposed here, as discussed above, is to improve fSNR in fMRI by integrating receive coils directly into the case device 1. One of the main fSNR limitations in conventional fMRI is the distance from the receive coils to the brain. Because conventional whole-head receive coils are generally manufactured to accommodate the majority of human heads, the coils are often quite far from the brain. It has recently been shown that flexible surface MRI coils provide higher fSNR than volume coils when imaging shallow structures near the cortical surface (Arias et al. 2014). Thus, in some embodiments, flexible coils could be integrated directly into the case device 1. In such examples, the coils can be placed as near as possible to each individual's brain, and could provide higher fSNR than can be obtained with conventional head coils.

All of these possibilities suggest that the customized case device could help alleviate many problems with current fMRI research as well as other research or procedures wherein patient positioning and stability is critical.

Example Process

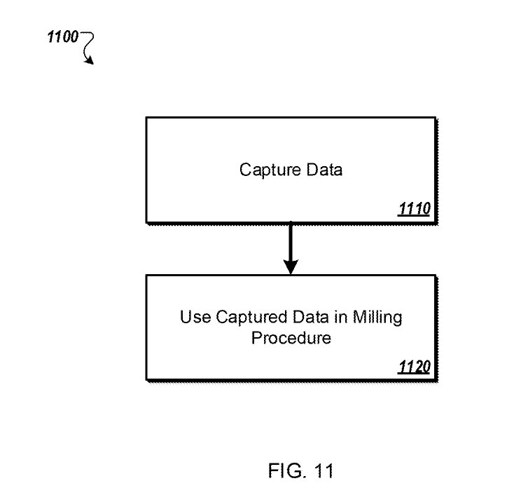

FIG. 11 is a flow diagram of an example process 1100 of forming a body positioning device for use in a medical procedure. In some implementations, the system 1000 of FIG. 10 can perform the process. In some implementations, the body positioning device can be the case device 1 of FIG. 1.

The process 1000 includes capturing data 1100 for a shape of a body part to be subjected to a medical procedure involving a machine that performs the medical procedure, and using the captured data 1120 in the performance of a milling procedure upon a source material to form a milled body positioning device having a milled inner surface with a shape configured to complement a shape of a body part to be subjected to a medical procedure.

In some implementations, the milled body positioning device can also include a connection structure adapted to mate with structure of a machine for performing the medical procedure so as to register the position of the body part within the milled body positioning device with the machine for performing the medical procedure. In some implementations, the body part can be a head, and the shape of the inner surface of the milled body positioning device can be configured to complement the shape of the head. In some implementations, the connection structure can be shaped to mate with a portion of the machine associated with a subject's head. In some implementations, the connection structure can have a shape that mates with the machine for performing the medical procedure, and the shape of the connection structure can be formed by a milling process based on data for a shape of the machine that performs the medical procedure. In some implementations, the body part can be a body part that includes bony structure near the surface of the skin. In some implementations, the medical procedure can be an imaging procedure and the machine can be an imaging machine. In some implementations, the connection structure can have a surface with a shape configured to complement the shape of imaging head coils of the imaging machine. In some implementations, the medical procedure can be a radiation therapy procedure and the machine can be a radiation therapy machine. In some implementations, the milled body positioning device can include multiple components adapted to be assembled so that the human head can be contained within an inner chamber of the milled body positioning device when assembled. In some implementations, the source material can be a material comprising one of polyurethane, expanded polystyrene, and other expanded polymer foam material.

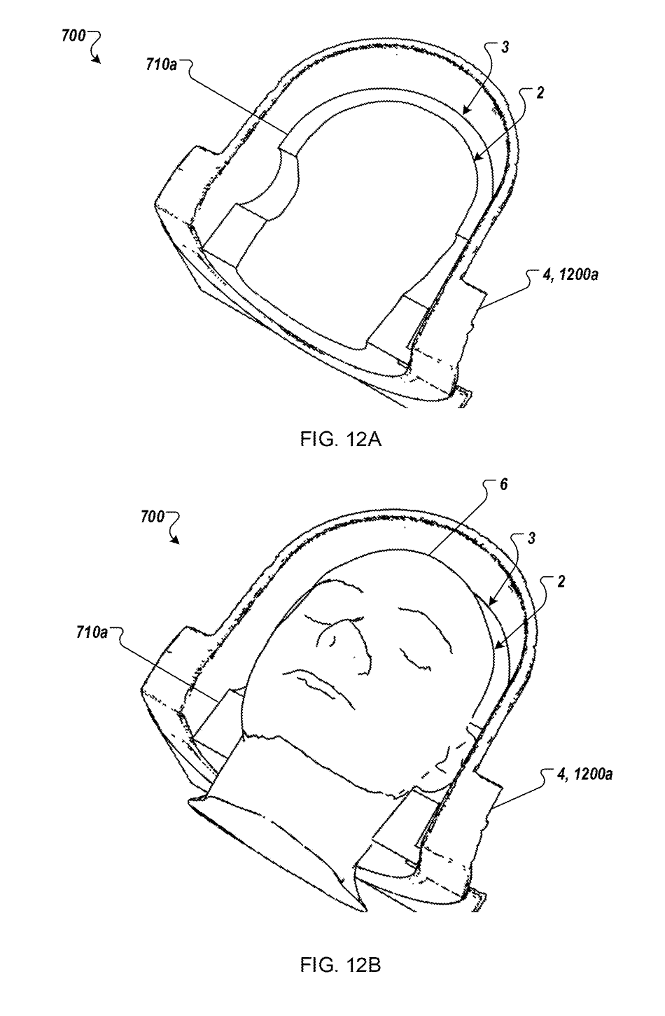

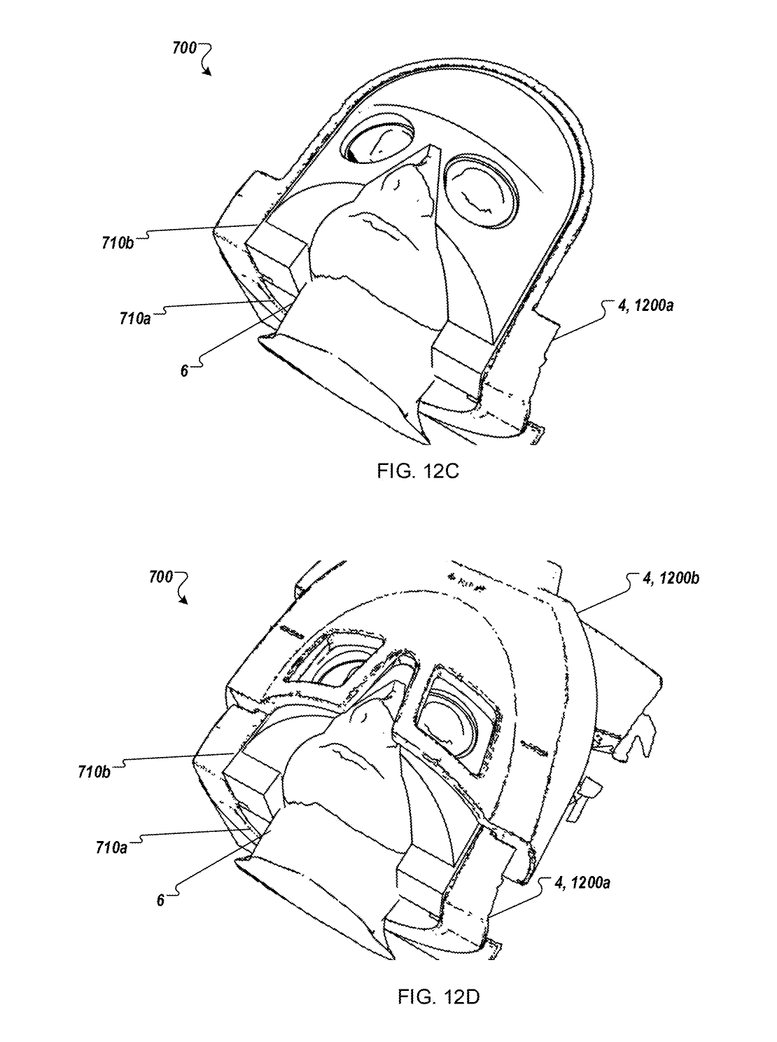

FIGS. 12A-12D show an example of the case device 700 in use. In some implementations, the case device 1 can be used in an appropriately corresponding manner. Referring now to FIG. 12A, the back portion 710a of the case device 700 is shown inserted into a back portion 1200a of the MRI head coil 4. The outer surface 3 of the back portion 710a is formed with a shape that closely matches that of the inner surface of the back portion 1200a. The inner surface 2 of the back portion 710a is formed with a shape that closely matches that of the back portion of the subject's head 6.

Referring now to FIG. 12b, the subject can be placed such that the subject's head 6 rests upon the inner surface 2. For example, the subject may lie down, resting the subject's head 6 in the back portion 710a (e.g., like a pillow). In other examples, the back portion 710a can be placed upon a portion of the subject's head 6 (e.g., like a helmet). The back of the subject's head 6 registers with the back portion 710a, and the back portion 710a registers with the back portion 1200a, to at least partly register the subject's head 6 with the MRI head coil 4. Is some implementations, the back portion 710a may be the only portion used or needed for a particular medical procedure.

Referring now to FIG. 12C, the front portion 710b is placed over the front of the subject's head 6. The outer surface 3 of the front portion 710b is formed with a shape that closely matches that of the inner surface of a portion 1200b of the MRI head coil 4. The inner surface 2 of the front portion 710b is formed with a shape that closely matches that of the front portion of the subject's head 6 (e.g., the subject's face).

Referring now to FIG. 12D, the front portion 1200b of the MRI head coil 4 is placed on the front portion 710b of the case device 700. As such, the subject's head 6 can be maintained in a substantially fixed position and location relative to the MRI head coil 4. In some implementations, the case device 700 can be removed and late re-applied upon the subject's head 6 to place the subject's head 6 back into substantially the same position and location relative to the MRI head coil 4 (e.g., for a procedure that involved multiple treatments using the same or similar medical equipment).

Although a few implementations have been described in detail above, other modifications are possible. For example, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

1. A body positioning device for use in a medical procedure, the device comprising:
a body-worn structure having an inner surface with a shape configured to complement a shape of a body part to be subjected to a medical procedure;
wherein the body-worn structure comprises connection structure adapted to mate with structure of a machine for performing the medical procedure so as to register the position of the body part wearing the body-worn structure with the machine for performing the medical procedure.

2. The body positioning device of claim 1, wherein the body part is a head, and the shape of the inner surface is configured to complement the shape of the head.

3. The body positioning device of claim 2, wherein the connection structure is shaped to mate with a portion of the machine associated with a subject's head.

4. The body positioning device of claim 1, wherein the body part is a body part that includes bony structure near the surface of the skin.

5. The body positioning device of claim 1, wherein the medical procedure is an imaging procedure and the machine is an imaging machine.

6. The body positioning device of claim 5, wherein the connecting structure has a surface with a shape configured to complement the shape of imaging head coils of the imaging machine.

7. The body positioning device of claim 1, wherein the medical procedure is a radiation therapy procedure and the machine is a radiation therapy machine.

8. The body positioning device of claim 2, wherein the body-worn structure comprises multiple components adapted to be assembled so that the head of the subject is contained within an inner chamber of the body-worn device when assembled.

9. The body positioning device of claim 1, wherein the body-worn structure is made of a material comprising one of a polyurethane, expanded polystyrene, and other expanded polymer foam material.

10. The body positioning device of claim 9, wherein the inner surface of the body-worn structure is formed by a milling machine based upon input information regarding the shape of the body part to be subjected to the medical procedure.

11. A method of forming a body positioning device for use in a medical procedure, the method comprising:
capturing data for a shape of a body part to be subjected to a medical procedure involving a machine that performs the medical procedure; and
using the captured data in the performance of a milling procedure upon a source material to form a milled body positioning device having a milled inner surface with a shape configured to complement a shape of a body part to be subjected to a medical procedure.

12. The method of claim 11, wherein the milled body positioning device further comprises a connection structure adapted to mate with structure of a machine for performing the medical procedure so as to register the position of the body part within the milled body positioning device with the machine for performing the medical procedure.

13. The method of claim 12, wherein the body part is a head, and the shape of the inner surface of the milled body positioning device is configured to complement the shape of the head.

14. The method of claim 13, wherein the connection structure is shaped to mate with a portion of the machine associated with a subject's head.

15. The method of claim 14, wherein the connection structure has a shape that mates with the machine for performing the medical procedure, and the shape of the connection structure is formed by a milling process based on data for a shape of the machine that performs the medical procedure.

16. The method of claim 11, wherein the body part is a body part that includes bony structure near the surface of the skin.

17. The method of claim 11, wherein the medical procedure is an imaging procedure and the machine is an imaging machine.

18. The method of claim 17, wherein the connection structure has a surface with a shape configured to complement the shape of imaging head coils of the imaging machine.

19. The method of claim 12, wherein the medical procedure is a radiation therapy procedure and the machine is a radiation therapy machine.

20. The method of claim 13, wherein the milled body positioning device comprises multiple components adapted to be assembled so that the human head is contained within an inner chamber of the milled body positioning device when assembled.

21. The method of claim 11,
wherein the source material is a material comprising one of polyurethane, expanded polystyrene, and other expanded polymer foam material.

22. A system for forming a body positioning device for use in a medical procedure, the system comprising:
a receiver adapted to receive image data taken for a patient that is descriptive of a shape of a body part for the patient; and
processing circuitry adapted to transform the image data into computer readable instructions to be provided to an automated milling machine to mill a source material into a body positioning device having a milled inner surface with a shape configured to complement the shape of the body part for the patient.

23. The system of claim 22, wherein the processing circuitry is further adapted to produce computer readable instructions to be provided to the automated milling machine to mill the source material to also have a connection structure adapted to mate with structure of a machine for performing the medical procedure so as to register the body part within the body positioning device with the machine for performing the medical procedure.

24. The system of claim 23, further comprising stored data usable by the processing circuitry to determine milling instructions for a shape of the connecting structure.

25. The system of claim 22, further comprising an automated milling machine.

26. The system of claim 22, wherein the body part is a human head, and the shape of the inner surface of the milled body positioning device is configured to complement the shape of a head.

27. The system of claim 23, wherein the connection structure is shaped to mate with a portion of the machine associated with a patient's head.

28. The system of claim 27, wherein the connection structure has a shape that mates with the machine for performing the medical procedure, and the shape of the connection structure is formed by a milling process based on data for a shape of the machine that performs the medical procedure.

29. The system of claim 22, wherein the body part is a body part that includes bony structure near the surface of the skin.

30. The system of claim 23, wherein the medical procedure is an imaging procedure and the machine is an imaging machine.

31. The system of claim 30, wherein the connecting structure has a surface with a shape configured to complement the shape of imaging head coils of the imaging machine.

32. The system of claim 23, wherein the medical procedure is a radiation therapy procedure and the machine is a radiation therapy machine.

33. The system of claim 26, wherein the body positioning device comprises multiple components adapted to be assembled so that the head of the patient is contained within an inner chamber of the body positioning device when assembled.
